(19) United States
(12) Patent Application Publication
(10) Pub. No.: US 2022/0369906 A1
(43) Pub. Date: Nov. 24, 2022

(54) ENDOSCOPIC VISUALIZATION DEVICE, SYSTEM, AND METHODS

(71) Applicant: BOSTON SCIENTIFIC SCIMED, INC., Maple Grove, MN (US)

(72) Inventors: CRAIG MICHAEL WILSON, Minneapolis, MN (US); LANCE ADAM FREESEMAN, Greenfield, MN (US); JAMES P. ROHL, Prescott, WI (US); AARON ABBOTT, Columbia Heights, MN (US); JOEL T. EGGERT, Plymouth, MN (US); JAMES A. KLOS, Bay City, WI (US); SANDRA L. WEEDA, Scandia, MN (US); NIRAJ PRASAD RAUNIYAR, Plymouth, MN (US)

(73) Assignee: BOSTON SCIENTIFIC SCIMED, INC., Maple Grove, MN (US)

(21) Appl. No.: 17/748,653

(22) Filed: May 19, 2022

Related U.S. Application Data
(60) Provisional application No. 63/190,957, filed on May 20, 2021.

(51) Int. Cl. A61B 1/00; A61B 1/06; A61B 1/05; A61B 5/107

(52) U.S. Cl. CPC A61B 1/00096; A61B 1/0676; A61B 1/05; A61B 5/1079; A61B 5/1076; A61B 1/0011

(57) ABSTRACT

Imaging devices, imaging systems, and methods are presented for determining distances, depths, and sizes of a viewed tissue or object through a visualization device. The device may include an elongated shaft and an imaging component. The imaging component may extend through the elongated shaft. The imaging component may have a lens and may be configured to capture an image of an area exterior of the elongated shaft in a field of view of the lens. A transparent cover may extend over the lens. The transparent cover may be configured to cause one or more identifiers to appear in the image. The imaging device may include or be used with a computing device to analyze image data of captured images.

### CROSS REFERENCE TO RELATED APPLICATIONS

This application claims the benefit of and priority to U.S. Provisional Patent Application Ser. No. 63/190,957 filed on May 20, 2021, the disclosure of which is incorporated herein by reference.

### TECHNICAL FIELD

The disclosure pertains to medical devices and more particularly to visualization medical devices for improved viewing within a body structure, and methods for using such medical devices. Namely, the disclosure pertains to endoscopic visualization devices with improved viewing capabilities.

### BACKGROUND

A wide variety of medical devices have been developed for medical use. Some of these devices include catheters, ablation devices, visualization devices, snares, and the like, and delivery devices and/or systems used for delivering such devices. These devices are manufactured by any one of a variety of different manufacturing methods and may be used according to any one of a variety of methods. Of the known medical devices, delivery systems, and methods, each has certain advantages and disadvantages. There is an ongoing need to provide alternative medical devices and delivery devices as well as alternative methods for manufacturing and using medical devices and delivery devices.

### SUMMARY

This disclosure provides design, material, manufacturing method, and use alternatives for medical devices.

A first example is a visualization medical device. The visualization medical device includes an elongated shaft, an imaging component extending through the elongated shaft, the imaging component having a lens and is configured to capture an image of an area exterior of the elongated shaft in a field of view of the lens, and a transparent cover extending over the lens, the transparent cover configured to cause one or more identifiers to appear in the image.

Alternatively or additionally to any of the examples above, a lighting component extends through the elongated shaft, the lighting component is configured to light the area exterior of the elongated shaft.

Alternatively or additionally to any of the examples above, the transparent cover includes a reticle in the field of view of the lens and the reticle appearing in a captured image is an identifier of the one or more identifiers.

Alternatively or additionally to any of the examples above, the reticle is a fixed marking on the transparent cover.

Alternatively or additionally to any of the examples above, the reticle comprises one or more liquid crystal display (LCD) elements actuatable to cause the one or more identifiers to appear in the image.

Alternatively or additionally to any of the examples above, a lighting component extends through the elongated shaft, the lighting component is configured to light the field of view of the lens and cause a shadow of the reticle to appears in the field of view of the lens.

Alternatively or additionally to any of the examples above, the transparent cover is an extension extending distally from a distal end of the elongated shaft.

Alternatively or additionally to any of the examples above, a distal end of the elongated shaft has one or more first mating components and a proximal side of the transparent cover has one or more second mating components configured to mate with the one or more first mating components.

Alternatively or additionally to any of the examples above, at least one of the one or more first mating components is a female mating component and at least one of the one or more second mating components is a male mating component configured to mate with the female mating component.

Alternatively or additionally to any of the examples above, the transparent cover is in direct contact with the lens.

Alternatively or additionally to any of the examples above, the transparent cover is a first color at a first location and a second color at a second location.

Another example is a method for providing anatomical distance measurements using a medical visualization device. The method includes applying a light through a transparent cover on an elongated shaft, the transparent cover having one or more markings and the light applied through the transparent cover causes a shadow to appear on a surface exterior of the transparent cover, capturing an image through the transparent cover, the image including a view of the one or markings and at least a portion of the shadow, and determining a measure of an anatomical distance in the image based on the view of the one or more markings and the portion of the shadow in the image.

Alternatively or additionally to any of the examples above, adjusting a distal end of the elongated shaft to cause a target object to be within a field of view of a lens through which the image is captured.

Alternatively or additionally to any of the examples above, determining a size measurement of an object in the image based on the measure of the anatomical distance.

Alternatively or additionally to any of the examples above, at least one of the one or more markings is an electronic component actuatable to be viewable through a lens used in capturing the image.

Another example is a visualization medical system. The visualization medical system includes an elongate tool having a transparent cover, the transparent cover having one or more markings thereon, an imaging component configured to capture an image through the transparent cover, the image including one or more identifiers representative of at least one of the one or more markings on the transparent cover, a video processor configured to analyze image data of the image that is received from the imaging component, and wherein the video processor is configured to determine one or more measurements of an anatomical distance in the image based on the analysis of the image data.

Alternatively or additionally to any of the examples above, a light source is configured to apply light through the transparent cover and cause a shadow of the one or more markings to appear in the image.

Alternatively or additionally to any of the examples above, the video processor is configured to determine the one or more measurements of the anatomical distance in the image based on the one or more markings in the image and the shadow of the one or more markings in the image.

Alternatively or additionally to any of the examples above, the one or more measurements of the anatomical distance in the image include determining a distance between the one or more markings on the transparent cover and a surface on which the shadow is located.

Alternatively or additionally to any of the examples above, the transparent cover is in direct contact with a lens of the imaging component.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a schematic perspective view of an illustrative visualization medical device;

FIG. 2 is a schematic view of an illustrative visualization medical system;

FIG. 3 is a perspective view of a distal end of an illustrative visualization medical device;

FIG. 4 is an end view of the distal end of the illustrative visualization medical device depicted in FIG. 3;

FIGS. 5-11 depict various views of an illustrative transparent cover for a visualization medical device;

FIG. 12 depicts a schematic view of an illustrative use of a visualization medical device;

FIG. 13 depicts a schematic view of an image taken with an illustrative visualization medical device; and

FIG. 14 is a schematic diagram of an illustrative method of using a visualization medical system.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

Visualization medical devices are used in numerous procedures including, but not limited to, atrial fibrillation ablation (e.g., electroporation), treatment of structural heart disease (e.g., valve repair), diagnostic evaluation of tissues, stone management, prostate management, gynecological surgery, etc. During these procedures, opaque fluids (e.g., blood and/or other opaque fluids) and/or other matter may present problems for direct visualization of target tissue, target objects, and target areas. Further, when viewing target tissue, objects, and areas in a patient body, it is beneficial to be able to accurately and quickly assess a distance between a distal end of an endoscopic visualization medical device (e.g., an endoscope and/or other suitable visualization medical device) and a target tissue, object and/or area, and one or more size dimensions of a target tissue or object in a field of view of the visualization medical device.

In some cases, a visualization medical device may include a balloon adjacent to and/or covering at least part of a distal end of the visualization medical device to facilitate a view through opaque fluids and/or to prevent particles from positioning between the visualization medical device and a target tissue or object. In such cases, the balloon may be a clear, highly compliant elastomer balloon that may be inflated using a saline solution to displace fluid and/or other material that may hinder a view through the visualization medical device. The balloon and saline used to inflate the balloon may create a path through the fluid and/or other material that otherwise hinders a view through the visualization device to allow visible light to be transmitted from a light source (e.g., fiber transmission cable, LED light, etc.) to a tissue and/or object of interest and for the issue and/or object to reflect light back to a receptor (e.g., camera, lens, fiber optic bundle, etc.) of the visualization medical device. However, such a configuration for a visualization medical device may have drawbacks including, but not limited to, the visualization medical device and/or an object in a field of view of the visualization medical device may displace undesirable or unpredictably in response to inflation of the balloon, the saline solution used to inflate the balloon may electrically compromise electronic components of the visualization medical device, the visualization pathway may be compromised if the balloon is torn or damaged, and as the saline solution typically used to inflate the balloon is output into the patient through weeping holes and the saline can cause damage to a patient if an excessive amount is output into the patient.

A transparent displacement extension or cover that does not require saline or other inflation fluid has been developed for use with or as part of the visualization medical device. The transparent cover may be permanently or removably attached to a visualization medical device so as to create displacement of fluid or materials between a lens or light receiving element of a visualization component and a target object or tissue. The transparent cover may be formed from a visible light transmitting and non-conductive elastomer material that is compliant enough to provide safe insertion through the vasculature or other body lumen or cavity, but is not malleable enough to result in inadvertent and/or undesirable deformation. Other configurations of the transparent cover are contemplated.

Visualization medical devices are often monocular and lack a direct technique to determine a depth and/or size of an object or other target in a field of view of the visualization component. To facilitate gauging sizes and/or depths (e.g., distances) of objects in the field of view, the transparent cover may include one or more markings or indicators (e.g., reticles and/or other suitable indicators) that appear in the field of view of the visualization component. Illustrative configurations of the indicators, the visualization component, and techniques for determining sizes and depths of items in a field of view are discussed below.

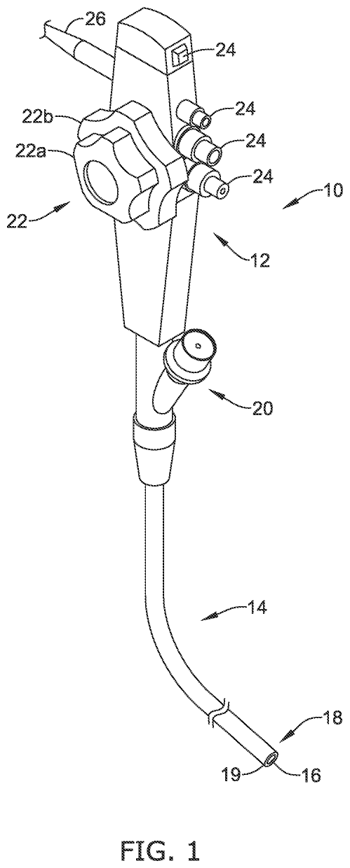

Turning to the Figures, FIG. 1 depicts an illustrative endoscopic visualization medical device 10 (e.g., an elongate tool such as an endoscope device and/or other suitable visualization medical device). The visualization medical device 10 may be any suitable type of visualization medical device usually identified by a particular anatomy desired to be reached. For example, the endoscopic visualization medical device 10 may be a bronchoscope, colonoscope, duodenoscope, esophagoscope, ureteroscope, nephroscope, guidetubes, introducers (with or without vision or visualization capabilities), or any other type of visualization medical device.

Among other components, the visualization medical device 10 may include a handpiece 12 and an elongated shaft 14 extending distally from the handpiece 12 to a distal end 19. The shaft 14 may include a lumen defining a working channel 16 extending through the shaft 14 from a distal end 19 of the shaft 14 to an access port 20 that may be positioned in the handpiece 12 or at another portion of the visualization medical device 10. The visualization medical device 10 may include one or more alternative and/or additional suitable features.

Although the visualization medical device 10 is depicted with a single working channel 16 in FIG. 1, it can be appreciated that in other embodiments, the visualization medical device 10 may include multiple working channels, as desired. The working channel(s) may be configured for the passage of a variety of surgical equipment, including, but not limited to, lighting, imaging devices and tools for irrigation, vacuum suctioning, biopsies, and drug delivery.

The handpiece 12 may include one or more controls 22, such as rotating knobs, which may be used to control movement of the distal end 19 of the shaft 14 during operation. For example, a first rotating knob 22a may control up and down movement or deflection of the distal end 19 of the shaft 14 in a first deflection plane, while a second rotating knob 22b may control side-to-side movement or deflection of the distal end 19 of the shaft 14 in a second deflection plane perpendicular to the first deflection plane. The handpiece 12 may also include one or more selectable buttons 24, which may be selectively actuated to activate suction, deliver fluid such as air, saline and/or water, etc. through a lumen of the visualization medical device 10, and/or perform other functions as desired. Additionally or alternatively, the handpiece 12 may include an optical cable 26 connected to an external light source (not shown). The optical cable 26 may extend from a distal end of the elongated shaft 14 through the elongated shaft to the external light source or energy source, but other configurations are contemplated. Other configurations and/or features of the handpiece 12 are contemplated.

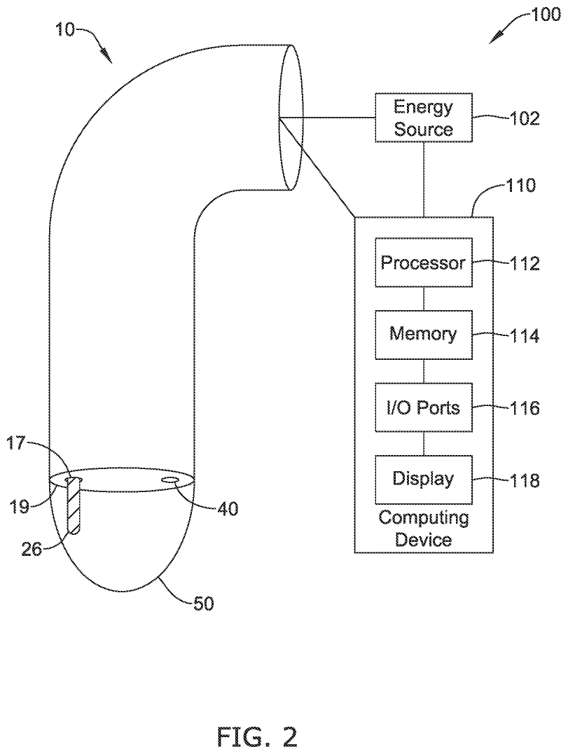

FIG. 2 schematically illustrates a system 100 (e.g., a visualization medical system) for and/or facilitating viewing and imaging a target area and/or object. In some cases, the system 100 may be configured to facilitate determining distances and/or sizes of objects in a field of view of an imaging component or system of the system 100.

The system 100 may include, among other components, the visualization medical device 10 and/or other suitable visualization medical devices having a transparent cover 50 (e.g., a transparent extension and/or other suitable transparent cover), an energy source 102, and a computing device 110. Although the visualization medical device 10, the energy source 102, and the computing device 110 are depicted separated from one another, the visualization medical device 10, the energy source 102, and the computing device 110 may be integrated in one another, as desired.

The energy source 102 may be any suitable type(s) of energy source. In one example, the energy source 102 may include an optical energy source (e.g., a holmium (Ho) laser source, a carbon dioxide (CO₂) laser source, an Argon laser source, a diode laser source, a high-power light emitting diode (LED), a pulsed light source, and/or one or more other suitable optical energy sources). In another example, the energy source 102 may be or may include line power, battery power, and/or one or more other suitable types of energy sources for powering an optical energy source and/or the computing device 110.

The energy source 102 may be in communication with and/or part of the visualization medical device 10 and/or the computing device 110. In one example, the energy source 102 may be mounted at the distal end 19 of the visualization medical device 10, or on a catheter or other suitable elongate member to facilitate lighting a target area or object for viewing through an imaging system or component. In some cases, the energy source 102 may be actuated and/or otherwise controlled using the computing device, but this is not required and the energy source 102 may be actuated and/or controlled in one or more other suitable manners.

The visualization medical system 100 may include and/or be in communication with one or more computing devices 110. The one or more computing devices 110 may be utilized to operate the visualization medical device 10, to control imaging with an imaging component or system of the visualization medical device 10, to operate one or more components of or used with the visualization medical device 10, to process data received from or obtained with imaging components or systems and/or other components of the visualization medical device 10, and/or utilized for one or more other suitable purposes.

Generally, the computing devices 110 may be and/or may be part of, for instance, a smart phone, a tablet, a personal digital assistant (PDA), a personal computer, a network device, a light source, a controller, a sensor, a switch, a remote server, a router, a modem, and/or other suitable computing device. However, configurations of the present disclosure are not limited to a particular type of computing device 110.

As depicted in FIG. 2, the computing devices 110 may include, among other suitable additional and/or alternative components, a processor 112, memory 114, input/output (I/O) ports 116, a display 118, and/or other suitable components. Although not shown, the computing device 110 may include a clock and/or a timer.

The processor 112 of the computing device 110 may include a single processor or more than one processor working individually or with one another (e.g., dual-core, etc.). The processor 112 may be configured to execute instructions, including instructions that may be loaded into the memory 114 and/or other suitable memory. Example processor components may include, but are not limited to, microprocessors, video processors, microcontrollers, multi-core processors, graphical processing units, digital signal processors, application specific integrated circuits (ASICs), field programmable gate arrays (FPGAs), discrete circuitry, and/or other suitable types of data processing devices.

The processor 112 may be or may include a video processor configured to facilitate analyzing image data that is received from an imaging system of or used with the visualization medical device 10 according to instructions stored in the memory 114. Such a video processor may be configured to determine one or more measurements of an anatomical distance and/or size of an object in an image or images captured by the imaging system based on an analysis of imaging data from the imaging system and/or determine one or more other suitable measurements.

The memory 114 may be any type of storage medium that can be accessed by the processor 112 to perform various examples of the present disclosure in an automated manner and/or in response to user inputs. For example, the memory 114 may be a non-transitory computer readable medium having computer readable instructions (e.g., computer or application program instructions, application program code of a mobile application or software, control algorithm software, and/or other suitable instructions) stored thereon that are executable by the processor 112 for performing one or more methods described herein.

The memory 114 may be volatile or nonvolatile memory. The memory 114 may also be removable (e.g., portable) memory, or non-removable (e.g., internal) memory. For example, the memory 18 may be random-access memory (RAM) (e.g., dynamic random access memory (DRAM) and/or phase change random access memory (PCRAM)), read-only memory (ROM) (e.g., electrically erasable programmable read-only memory (EEPROM) and/or compact-disk read-only memory (CD-ROM)), flash memory, a laser disk, a digital versatile disk (DVD) or other optical disk storage, and/or a magnetic medium such as magnetic cassettes, tapes, or disks, among other types of memory.

Further, although the memory 114 is illustrated as being located in the computing device 110, embodiments of the present disclosure are not so limited. For example, the memory 114 may also be located internal to another computing resource (e.g., enabling computer readable instructions to be downloaded over the Internet or another wired or wireless connection).

The I/O unit or ports 116 may be and/or may include any type of communication port(s) and may facilitate wired and/or wireless communication with one or more networks. In one example, the I/O port 116 may facilitate communication with one or more networks and/or other devices through any suitable connection including, but not limited to, radio communication, Ethernet, cellular communication, ZigBee®, REDLINK™, Bluetooth, Bluetooth Low Energy (BLE), WiFi, IrDA, dedicated short range communication (DSRC), EnOcean®, Near Field Communication (NFC), and/or any other suitable common or proprietary wired or wireless protocol. In one example, the I/O port 116 may at least include a port configured to communicate over the network with one or more components of or in communication with visualization medical system 100.

Although not particularly depicted, the computing device 110 may include a user interface. When provided, the user interface may be any suitable user interface and/or user interface components configured to facilitate a user of the computing device 110 interacting with the computing device 110 via the user interface. For example, the user interface may be used to provide information to and receive information from the user of the computing device 110 and/or the visualization medical system 100. For instance, the user interface may display images and/or video from the visualization medical device 10, display distances in the images and/or video, display lines in the images and/or video, display imaging and/or positioning data, display energy source data, and/or display other suitable data related to use of the visualization medical system 100. The user interface may include a keyboard or keyboard functionality, a pointer (e.g., a mouse, touchpad, or touch ball) or pointer functionality, a microphone, a speaker, a light system, a haptic system, a camera, a video camera, selectable buttons, adjustable dials, and/or other suitable user interface features the user may use to input information into and/or receive information from the computing device 110. Configurations of the present disclosure, however, are not limited to a particular type(s) of user interface.

The user interface may include a graphical user interface (GUI) that may have a display 118 (e.g., a screen) that may provide and/or receive information to and/or from the user of the computing device 110 (e.g., the display may display images captured using the visualization medical device 10 and/or data related to the images). The display 118 may be, for instance, a touchscreen (e.g., the GUI may include touchscreen capabilities). In some cases, the user interface may be part of another device (e.g. cell phone, remote work station) that is in wireless communication with the computing device 110.

In some cases, the visualization medical system 100 may further include one or more optical fibers 26 that may extend from the distal end 19 of the visualization medical device 10 through a lumen 17. The optical fibers 26 may be configured to deliver light energy from the energy source 102 to a treatment location. In other examples, the optical fiber 26 be fixed at the distal end 19.

An imaging device or component 40 may be used with or as a part of the visualization medical system 100 and may extend through the elongated shaft 14. The imaging component 40 may include a lens and may be configured with respect to the elongated shaft 14 such that it is able to capture an image of an area exterior of the elongated shaft 11 in a field of view of a lens. In one example, the lens may be located at the distal end 19 of the elongated shaft and a target area exterior of the distal end of the elongated shaft 14 may be within a field of view of the lens. The imaging component 40 may be considered an imaging system when including or used with a computing device (e.g., the computing device 110 and/or other suitable computing device).

The imaging component 40 may include any suitable device configured to provide images to the computing device 110 (e.g., the processor 112 of the computing device 110 for display on the display 118). In one example, the imaging component 40 may be or include a CMOS imaging sensor or other solid-state device or camera and one or more glass or polymeric lenses that produce electronic image signals representative of an image of the tissue or other objects in a field of view of the imaging component 40 (e.g., in a field of view of a lens of the imaging component 40). Although not required, the imaging component 40 may be or include a low light sensitive, low noise video VGA, CMOS, color imager or higher resolution sensor such as SVGA, SXGA, or XGA. In some examples, the imaging component 40 and/or a light source (e.g., the energy source and/or the optical fiber 26) may be fixed relative to one another at the distal end of the visualization medical device 10.

The transparent cover 50 may be configured to cover or extend over at least a lens or distal end portion (e.g., the lens, a distal end of an optical fiber, etc.) of the imaging component 40 and extend outward (e.g., distally when at the distal end 19 of the visualization medical device 10) from the lens or distal end portion of the imaging component 40. The transparent cover 50 may be configured to create a transparent space between the lens or distal end portion of the imaging component 40 and a target area or object in the field of view of the imaging component 40. In one example of the visualization medical device 10 being used in a blood field, the transparent cover 50 may displace opaque blood and allow for a clear view of a target area or object through the transparent cover.

When extending over the lens or distal end portion of the imaging component 40, the transparent cover 50 may be in direct contact with the lens or the distal end portion of the imaging component 40. Placing the lens or the distal end portion of the imaging component 40 in direct contact with the material of the transparent cover 50 improves a clarity of an image captured therethrough due to limiting a number of materials (e.g., materials that may have different refractive indices) that reflected light from the field of view of the lens or imaging component 40 has to pass through and thus, limits, mitigates, or prevents unnecessary image distortion.

The transparent cover 50 may be fixedly or removably attached to the visualization medical device 10 in one or more manners. Example techniques for affixing the transparent cover 50 to the visualization medical device 10 include, but are not limited to, overmolding, adhering with a medical adhesive, using plasma treatment methods, and/or other suitable affixing techniques. In one example, the transparent cover 50 may be molded onto a distal end 19 of the visualization medical device 10 in a fixed manner, but this is not required. When fixedly attached to the visualization medical device 10, the connection between the visualization medical device 10 and the transparent cover 50 may prevent fluid ingress. In some cases, the transparent cover 50 may be releasably attached via a snap connection, a threaded connection, a luer lock connection, and/or other suitable type of connection.

The transparent cover 50 may be made from any suitable type of material. In one example, the transparent cover may be made from a visible light transmitting elastomer material that is compliant enough to provide safe insertion through a patient's vasculature and/or through other patient delivery paths, yet not so malleable as to be subject to deformation when engaging patient tissue or objects at a target location. Further, the materials used for the transparent cover may be non-conductive so as to provide insulating properties to an electronic component in contact with the transparent cover 50 without need for other passivation materials. Example material for the transparent cover 50 may include, but are not limited to, silicone, polyurethane, soft urethanes, polyethylene terephthalate (PET), acrylic, polycarbonate, epoxy, and/or other suitable materials.

In one example of the transparent cover 50 on the visualization medical device 10, a silicone elastomer may be molded over the distal end 19 of the elongated shaft 14. Although not required, contact surfaces of the transparent cover 50 and/or the elongated shaft 14 may be treated with a primer or plasma treatment and a glue may be applied therebetween to promote adhesion between the parts. Additionally or alternatively, no glue or primer is utilized at certain locations to mitigate issues with indices of refraction of various materials in a field of view of the imaging component 40 or lens thereof.

The transparent cover 50 may have any suitable size or configuration. In some cases, the transparent cover 50 may have a general dome shape, but this is not required. Further, the transparent cover 50 may be configured to extend a distance from a distal end of the imaging component 40 and/or from the lens thereof at least a distance of a minimum focal length of the imaging component 40 and less than a maximum focal length of the imaging component 40, such that a clear path is provided distal of the imaging component 40 to a location at which the imaging component 40 can focus.

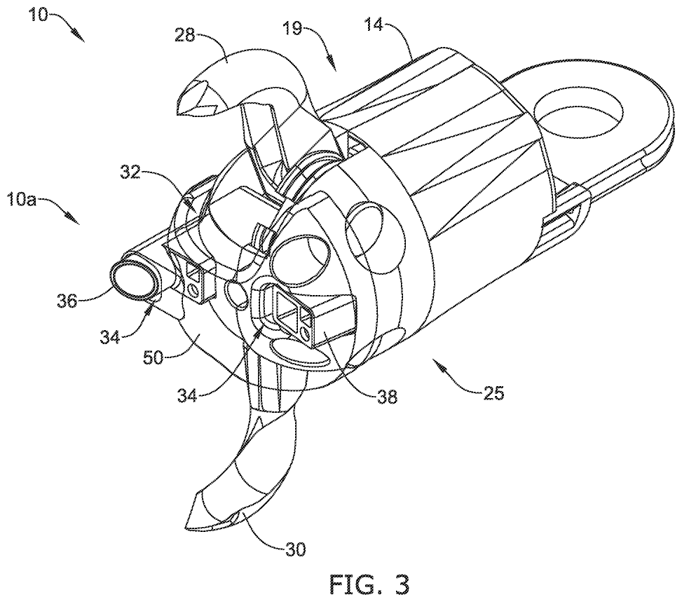

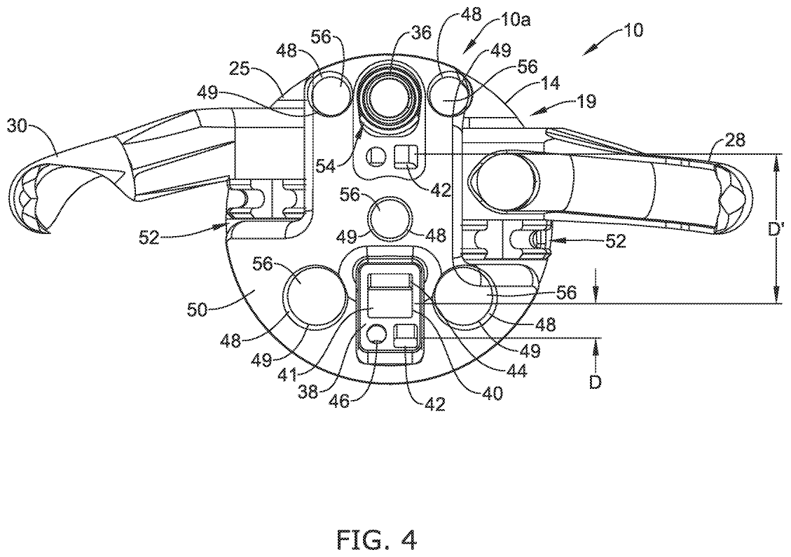

FIGS. 3 and 4 depict an illustrative visualization medical device 10 configured for use in an anchor delivery system for repairing a heart valve, where the visualization medical device includes a configuration of the transparent cover 50. An example visualization medical device 10 configured for use in an anchor delivery system for repairing a heart valve is described in U.S. patent application Ser. No. 16/104,085, titled ANCHOR DELIVERY SYSTEM AND METHODS FOR VALVE REPAIR filed on Aug. 16, 2018, which is hereby incorporated by reference in its entirety. Although the visualization medical device depicted in FIGS. 3 and 4 is configured for use in repairing a heart valve, the transparent cover 50 may be used with visualization medical devices 10 configured for additional and/or alternative purposes.

FIG. 3 illustrates a perspective view of a distal end 10a of the visualization medical device 10. The visualization medical device 10 depicted in FIG. 3 may include, among other components, a head 25, one or more pincer arms 28, 30 (depicted in an opened position), a stabilizer 36, a vision insert 38, and the cover 50. The pincers 28, 30 may be actuated from one or more controls located at a proximal end of the visualization medical device 10. In some cases, the visualization medical device 10 may include an elongated shaft (e.g., the elongated shaft 14 and/or other suitable elongated shaft) having one or more lumens configured to communicate with one or more holes or openings in the head 25.

The pincer arms 28, 30 may be disposed on the head 25 of the visualization medical device 10. A first pincer arms 28 may be disposed on the visualization medical device 10 at a location directly or substantially opposite from a second pincer arm 30, but this is not required. In some cases, the pincer arms 28, 30 may be actuatable (e.g., rotatable and/or adjustable in one or more other suitable manners) between an opened position and a closed position.

The stabilizer 36 may be any suitable configuration configured to stabilize the distal end 10a of the visualization medical device 10. In some cases, the stabilizer 36 may be helical (e.g., a helical anchor), but this is not required. The stabilizer 36 may be at a location such that the stabilizer 36 does not impede movement of the pincer arms 28, 30. The stabilizer 36 may be actuated using one or more controls at a proximal end of the visualization medical device 10.

As depicted in FIG. 3, the transparent cover 50 may extend from and/or be attached to a distal end of the head 25. The transparent cover 50 may have one or more gaps 52, holes 54, and/or other suitable configurations to facilitate a function and/or purpose of the visualization medical device 10, as described in greater detail below, but this is not required.

FIG. 4 depicts an end view of the illustrative visualization medical device 10 depicted in FIG. 3. Similar to as shown in FIG. 3, the pincer arms 28, 30 are depicted in an opened position to facilitate viewing components at the distal end 10a of the visualization medical device 10.

The visualization insert 38 may be located at a side of the visualization medical device 10 substantially opposite of the stabilization component 36, but this is not required and the visualization insert 38 may be located at one or more alternative or additional locations along the visualization medical device 10. In some cases, the visualization insert 38 may be disposed such that a target area distal of the distal end 10a of the visualization medical device 10 may be monitored by the imaging component 40 (e.g., a lens 41 or other imaging component and/or imaging system), a light 42, and a sensor 44.

Further, the imaging insert 38 may include one or more ports 46, which may be in fluid communication with a fluid lumen extending through the visualization medical device 10 and configured to allow fluid to be output therethrough for flushing a target area distal of the visualization medical device 10. When so configured, the transparent cover 50 may have an opening to allow fluid to be output from one or more of the ports 46. Alternatively or additionally, one or more of the port(s) 46 may be located at one or more other locations adjacent the distal end 10a of the visualization medical device 10, as the transparent cover 50 may extend over an imaging insert 38.

The imaging component 40 may be positioned at the distal end 10a of the visualization medical device 10 so as to have a field of view that allows a user of the device 10 to view a target location distal of the visualization medical device 10. In one example, the imaging component 40 or lens thereof may a field of view of approximately ten (10) to (16) millimeters in diameter. Although the imaging component 40 is depicted as having a single lens 41, it is contemplated that the imaging component 40 may include one or more lenses.

The light(s) 42 may be any suitable type of light and more than one light may be utilized, as desired. The light(s) 42 may be configured from the optical fiber 26 connected to an energy or light source and/or one or more other suitable lighting configurations. In one example, the light 42 may be or may include a light emitting diode (LED) and/or one or more other suitable types of lights. As depicted in FIG. 4, a single light 42 may be utilized adjacent the imaging component 40 and a single light 42 may be utilized adjacent the stabilizer 36. A dual light system may facilitate providing better light for capturing images, viewing a target area, and/or analyzing image data of the target area, but other lighting configurations are contemplated. In some cases, the light(s) 42 may be known distances D, D′ from the imaging component 40 or otherwise have known positions relative to the imaging component 40.

The sensors 44 may be any suitable type of sensors 44 configured to facilitate one or more functions of the visualization medical device. Example sensor types include, but are not limited to, ultrasonic sensors and degree of freedom (DOF) sensors. The ultrasonic sensor(s) may be utilized to verify a location of the distal end 10a of the visualization medical device 10 and/or may be utilized for one or more other suitable purposes. The DOF sensor(s) may be utilized to sense movement of the visualization medical device 10 and/or may be utilized for one or more other suitable purposes.

The imaging component 40, the light(s) 42, the sensor(s) 44, and/or the ports 46 may be in communication with one or more controllers and/or computing devices 110 at or in communication with a proximal end of the of the visualization medical device 10. Further, the imaging component 40, the light(s), the sensor(s) 44, and/or the ports 46 may be controlled in an automated manner using a predetermined procedure stored in one of the computing devices 110 and/or may be controlled entirely or at least partially in a manual manner by interacting with the computing device 110 and/or one or more other suitable actuator or control mechanism.

As depicted in FIG. 4, the transparent cover 50 overlays or is otherwise located at the distal end 10a of the visualization medical device 10 and may include one or more gaps 52. With respect to the configuration of the visualization medical device 10 depicted in FIGS. 3 and 4, the gaps 52 in the transparent cover 50 may be utilized to facilitate use of one or more features at the distal end 10a of the visualization medical device 10. In one example, the transparent cover 50 may include one or more gaps 52 configured to facilitate receiving the pincer arms 28, 30 and allowing the pincer arms 28, 30 to move along their respective ranges of motion.

As depicted in FIG. 4, the transparent cover 50 may include one or more openings 34. Similar to the gaps 52, the openings 54 may be positioned within the transparent cover 50 and/or sized to facilitate use of one or more features at the distal end 10a of the visualization medical device 10. In one example, the transparent cover 50 may include one or more openings 54 configured to allow use of and/or facilitate receiving the stabilizer 36, to allow for excretion of fluid from ports 46, and/or other components of the visualization medical device 10.

Although the transparent cover 50 is depicted with gaps 52 and openings 54 to accommodate features at the distal end 10a of the visualization medical device, the transparent cover 50 may have one or more other suitable configurations to accommodate features at the distal end 10a of the visualization medical device 10. In one example, the transparent cover 50 may extend over at least part of the imaging insert 38 so as to cover one or more of the imaging component 40, the lighting 42, and the sensors 44. When the transparent cover 50 extends over one or both of the imaging component 40 and the lighting 42, a suitable material for the transparent cover 50 may be a silicone elastomer, which has a refraction index (RI) of 1.1, and/or other transparent materials having suitable RI values.

In some cases, the distal end 10a of the visualization medical device 10 (e.g., the distal end 19 of the elongated shaft 14) may include one or more mating features 48 (e.g., first mating features), as viewed through the transparent cover 50 in FIG. 4, configured to increase contact surface area between the distal end 19 of the elongated shaft 14 of the visualization medical device 10 and a proximal side or mating side of the transparent cover 50 in a manner that promotes a connection there between. The mating features 48 of the visualization medical device 10 may be any suitable type of mating features. For example, the mating features 48 may be male mating features, female mating features, hooks, buttons, snaps, and/or other suitable types of mating features. In one example, the mating features of the visualization medical device may be female mating features 49, but this is not required, and male mating features 56 (e.g., second mating features) of the proximal side of the transparent cover 50 may engage the female mating features 49 of the visualization medical device 10.

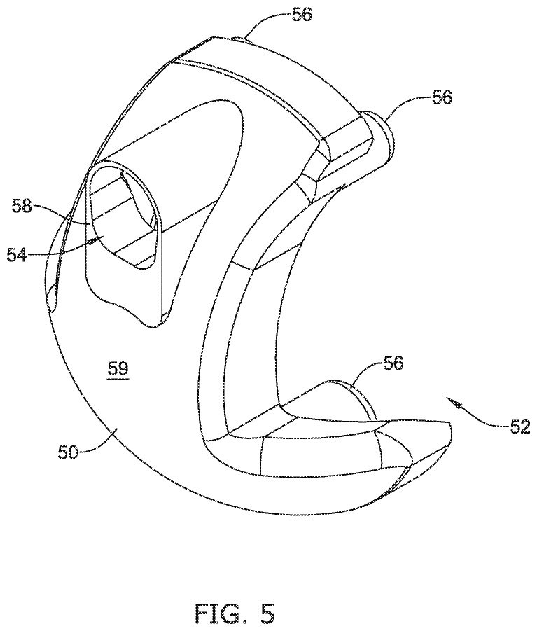

FIGS. 5-11 depict various views of the illustrative transparent cover 50 for use with the visualization medical device 10 depicted in FIGS. 3 and 4. Other configurations of the transparent cover 50 are contemplated.

FIG. 5 is a perspective view of the illustrative transparent cover 50 depicted in FIGS. 3 and 4. As shown, the transparent cover 50 may include the gap 52 for receiving a pincer arm (e.g., the pincer arm 28 and/or other suitable pincer arm), male mating components 56 configured to mate with the female mating components 49 of the distal end 19 of the elongated shaft 14, and the opening 54 for receiving the stabilizer 36 and/or features adjacent thereto of the visualization medical device 10. As depicted in FIG. 5, the opening 54 for receiving the stabilizer 36 may be an extension or protrusion 58 from an otherwise dome-like front surface 59 (e.g., a distal surface) of the transparent cover 50, but other suitable configurations are contemplated. The front surface 59 may be generally convex, but this is not required.

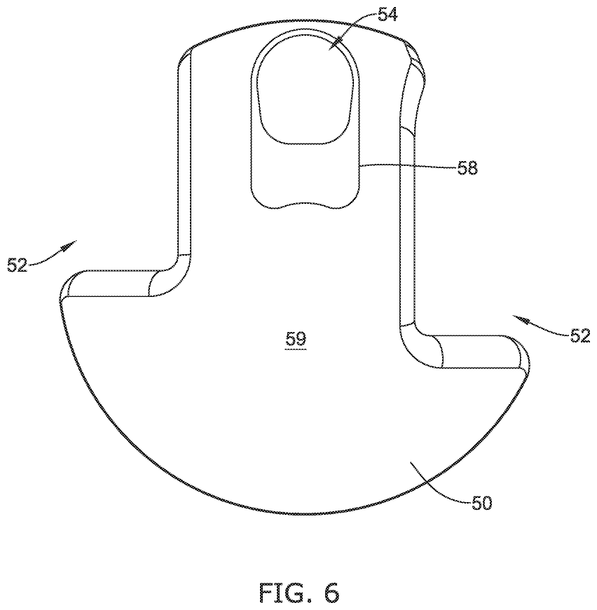

FIG. 6 is a front view of the illustrative transparent cover 50 depicted in FIGS. 3 and 4. As shown, the transparent cover 50 may include the gaps 52 for receiving pincer arms (e.g., the pincer arm 28, the pincer arm 30, and/or other suitable pincer arms) and the protrusion 58 defining the opening 54 for receiving the stabilizer 36 and/or features adjacent thereto of the visualization medical device 10.

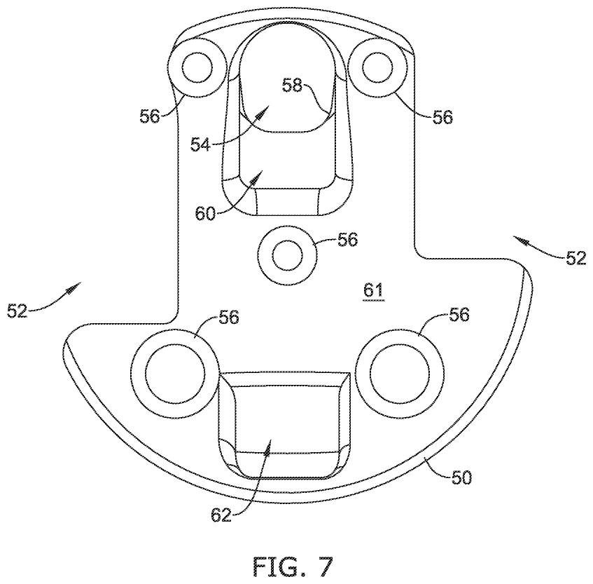

FIG. 7 is a back view of the illustrative transparent cover 50 depicted in FIGS. 3 and 4. As shown, the transparent cover 50 may include the gaps 52 for receiving pincer arms (e.g., the pincer arm 28, the pincer arm 30, and/or other suitable pincer arms), male mating components 56 configured to mate with the female mating components 49 of the distal end 19 of the elongated shaft 14, and the protrusion 58 defining the opening 54 for receiving the stabilizer 36 and/or features adjacent thereto of the visualization medical device 10. Further, the protrusion 58 may at least partially define a recess 60 in a back surface 61 of transparent cover 50 opposite the front surface 59, where the recess 60 may be configured to receive at least part of the stabilizer 36 and/or components adjacent the stabilizer. In some cases, a recess 62 in the back surface 61 may be configured to receive one or more other components of the visualization medical device 10, for example, the imaging insert 38 and associated components. The back surface 61 may be generally concave, but this is not required.

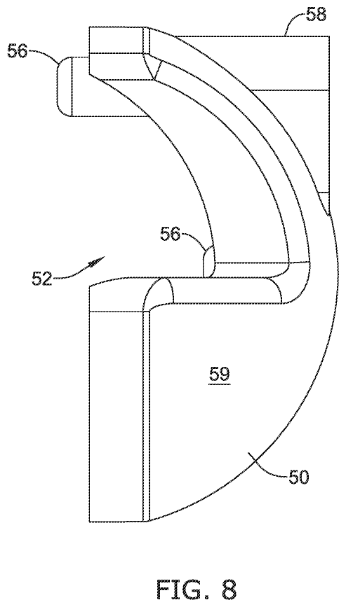

FIG. 8 is a first side view of the illustrative transparent cover 50 depicted in FIGS. 3 and 4. As shown, the transparent cover 50 may include the gap 52 for receiving a pincer arm (e.g., the pincer arm 30 and/or other suitable pincer arm), the male mating components 56 configured to mate with the female mating components 49 of the distal end 19 of the elongated shaft 14, and the protrusion 58 extending from the front surface 59 for receiving the stabilizer 36 and/or features adjacent thereto of the visualization medical device 10.

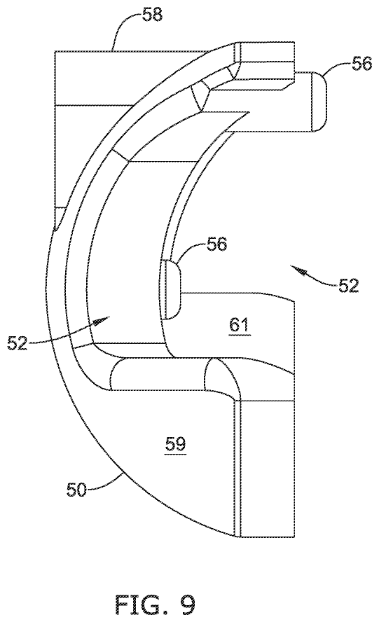

FIG. 9 is a second side view, opposite of the first side view, of the illustrative transparent cover 50 depicted in FIGS. 3 and 4. As shown, the transparent cover 50 may include the gaps 52 for receiving pincer arms (e.g., the pincer arm 28, the pincer arm 30 and/or other suitable pincer arms), the male mating components 56 configured to mate with the female mating components 49 of the distal end 19 of the elongated shaft 14, and the protrusion 58 extending from the front surface 59 for receiving the stabilizer 36 and/or features adjacent thereto of the visualization medical device 10.

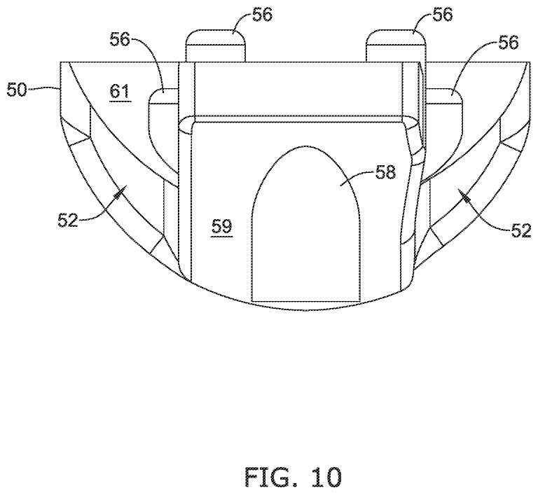

FIG. 10 is a top side view of the illustrative transparent cover 50 depicted in FIGS. 3 and 4. As shown, the transparent cover 50 may include the gaps 52 for receiving pincer arms (e.g., the pincer arm 28, the pincer arm 30 and/or other suitable pincer arms), the male mating components 56 configured to mate with the female mating components 49 of the distal end 19 of the elongated shaft 14, and the protrusion 58 extending from the front surface 59 for receiving the stabilizer 36 and/or features adjacent thereto of the visualization medical device 10.

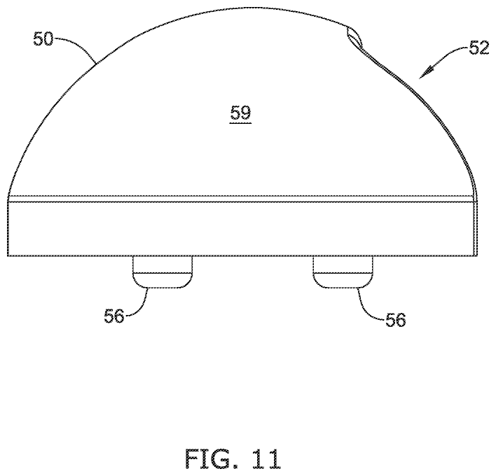

FIG. 11 is a bottom side view of the illustrative transparent cover 50 depicted in FIGS. 3 and 4. As shown, the transparent cover 50 may include the gap 52 for receiving a pincer arm (e.g., the pincer arm 28 and/or other suitable pincer arm) and the male mating components 56 configured to mate with the female mating components 49 of the distal end 19 of the elongated shaft 14.

Although not depicted, one or more portions of the transparent cover 50 may be colored, while still allowing light to pass therethrough. In one example, a portion of the transparent cover 50 overlaying the lights 42 may be a first color causing the light from the lights 42 to change a color from a natural color for the purpose of improving a view of a target area relative to a fluid (e.g., blood and/or other suitable fluid) at or adjacent the target area and a portion of the transparent cover 50 overlaying the imaging component may be second color. Example colors include, but are not limited to, no color, blue, green, etc. The color of various portions of the transparent cover 50 may be manipulated by addition of phosphorescents/colorants to the material of the transparent cover 50 during manufacturing of transparent cover 50 and/or manipulated in one or more other suitable manners.

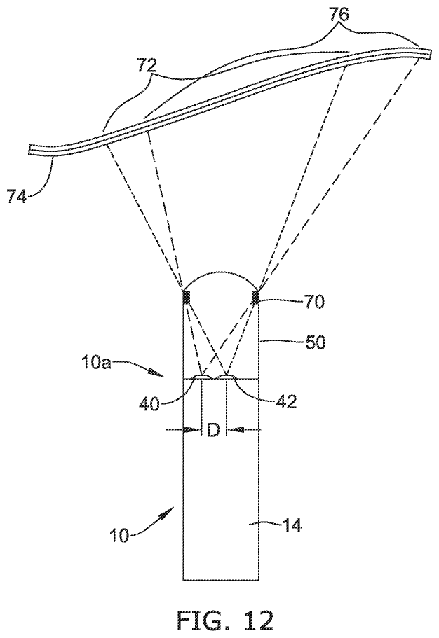

FIG. 12 depicts a schematic view of an illustrative visualization medical device 10 having the elongated shaft 14 with the transparent cover 50 located at the distal end 10a of the visualization medical device 10, where the transparent cover 50 includes one or more markings 70 (e.g., reticles and/or other suitable markings) positioned thereon. The markings 70 may be positioned in the transparent cover 50 so as to cause one or more identifiers to appear in an image of an area exterior of the visualization medical device taken through the transparent cover 50 using the imaging component 40. In some cases, the markings 70 may be configured to block light.

The markings 70 may be positioned on and/or in the transparent cover 50 to facilitate a user of the visualization medical device 10 gauging sizes and/or distances of surfaces and/or objects in a field of view of the imaging component 40 (e.g., the lens). Further, the markings 70 may be utilized to facilitate positioning the visualization medical device 10 at a desired location with respect to target tissue and/or one or more target objects in the field of view of the imaging component 40.

The markings 70 may be any suitable type of marking component. In one example, the markings 70 may be formed from one or more structures embedded in the transparent cover, so as to be fixed on or in the transparent cover 50. Alternatively or in addition, one or more of the markings 70 may be a liquid crystal display (LCD) component that may be selectively and electronically actuated to cause an indicator representative of the LCD component to appear, or not, in a view or image through the imaging component 40.

The markings 70 may have any suitable shape and/or size. Example shapes of the markings 70 may include, but are not limited to, circles, crosshairs, dashes, etc.

The markings 70 may be positioned at any suitable location in and/or along the transparent cover 50. In one example, the markings 70 may be positioned in the transparent cover 50 such that an entirety of or at least a portion of the markings 70 are positioned within the field of view of the imaging component 40 and are within a light path from the lights 42. Such a positioning of the markings 70 may facilitate using the markings 70 in analyzing an image through the imaging component 40 and/or positioning the visualization medical device 10 at a desired location with respect to a target location and/or a target object. Other suitable configurations of the markings 70 are contemplated.

When the markings 70 are positioned in the transparent cover 50 such that an entirety of or at least a portion of the markings 70 are positioned within the field of view of the imaging component 40 and are within a light path from the lights 42, the light path from the lights 42 may cross the markings 70 and cause a markings shadow 72 to appear on patient tissue 74 at least partially within a field of view 76 of the imaging component 40. In such cases, an image taken through the imaging component 40 may include indicators, which may be a captured view of the marking(s) 70 and/or at least a portion of the markings shadow 72.

As discussed below, such an image (e.g., image data) may be analyzed by a computing device (e.g., in real time with results displayed on a screen and/or the image) to determine a distance between the distal end 10a of the visualization medical device 10 and the patient tissue 74 and/or a size of an object in the image. Additionally and/or alternatively, a user of the visualization medical device 10 may be able to infer in real time a distance between the distal end 10a of the visualization medical device 10 and the patient tissue 74 and/or a size of an object in the image by viewing the markings shadow 72, particularly when the markings shadow 72 is viewed relative to the marking 70.

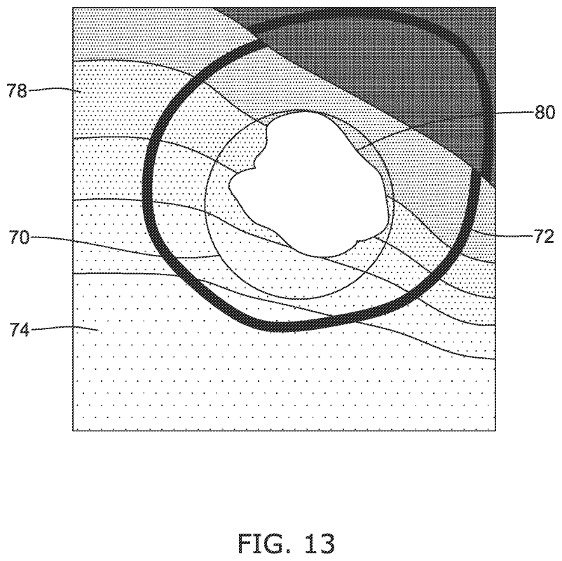

FIG. 13 depicts an image 78 through an imaging component (e.g., the imaging component 40 and/or other suitable imaging component) of a visualization medical device (e.g., the visualization medical device 10 and/or other suitable visualization medical devices). The image 78 depicts patient tissue 74 and an object 80 (e.g., a target stone to be removed) within the marking 70, where a light path from a light source (e.g., the lights 42) encounters the circular marking 70 causing the markings shadow 72 to appears on the patient tissue 74 and at least partially in the image 78 (e.g., an entirety of the shadow 72 appears in the image 78 of FIG. 13).

Although the image 78 depicts a shading of the patient tissue 74 to show a contour thereof, that contour of the patient tissue 74 may be difficult to ascertain in the image 78 without the use of a marking, the markings shadow, and/or a second device. As can be seen, the markings shadow 72 on the patient tissue changes shape with the contour and thus, allows a user viewing the shadow to understand the patient tissue 74 in the upper right of the image 78 may be closer to the imaging component 40 than the patient tissue 74 in the lower left of the image 78. Further, the user and/or a computing device may utilize the marking 70, the markings shadow 72 and/or a known distance (e.g., D, D′ and/or other suitable known distance) between the imaging component 40 and the lights 42 to determine distances and/or sizes of tissue and/or objects in the image 78.

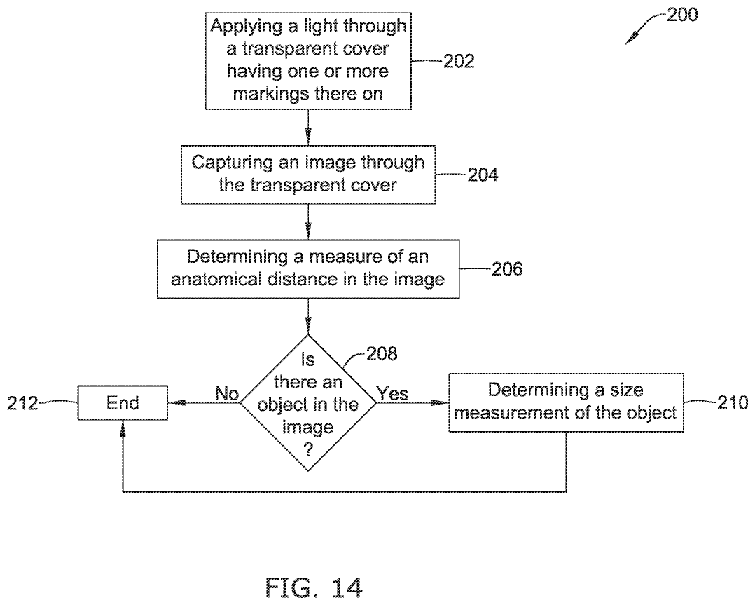

FIG. 14 depicts a schematic method 200 of determining distances and/or sizes of tissue and/or objects in an image captured using a visualization medical device (e.g., the visualization medical device 10 and/or other suitable visualization medical device). The method 200 may be automated and performed entirely within a computer and/or at least partially using a computer. In some cases, the method 200 may be saved as a set of instructions stored in memory and executable by a processor of the computing device. Alternatively or in addition, the method 200 may be executed in a manual manner and/or in response to a manual actuation.

The method 200 may include applying 202 a light path (e.g., via the lights 42 and/or other suitable light source) through a transparent extension or cover (e.g., the transparent cover 50 and/or other suitable transparent cover) having one or more markings thereon (e.g., the markings 70 and/or other suitable markings). Although not required, the lights producing the light path may be located at a distal end of the visualization medical device and the transparent cover may extend over at least part of the lights. The light path may be positioned relative to the one or more markings and/or the transparent cover, such that at least a portion of the light path is blocked by the markings and casts a markings shadow (e.g., the markings shadow 72 and/or other suitable shadow) on patient tissue.

Using an imaging device (e.g., the imaging component 40 and/or one or more other suitable imaging devices) of the visualization medical device, the method 200 may include capturing 204 an image through the transparent cover extending over or covering the imaging device. The captured image may include a view of patient tissue, optionally—an object in the patient, a marking on the transparent cover, and a shadow of the marking. In some cases, the distal end of the visualization medical device may be positioned and/or adjusted to cause an object (e.g., a target object and/or other suitable object) to be within a field of view of the imaging device (e.g., a field of view of the lens of the imaging device) to ensure the object in the patient appears in the captured image.

The method 200 may further include determining 206 a measure of an anatomical distance (e.g., depths, etc.) in the image. In some cases, the measure of the anatomical distance in the image may be determined based, at least in part on, the shadow in the image, the markings in the image, and/or a known (e.g., predetermined) distance between the imaging device, the markings, and a light. In one example, a computing device (e.g., the processor 112, such as a video processor, of the computing device 110 and/or other suitable computing devices) may analyze image data of the image to identify the identifiers representing markings on the transparent cover and the marking shadows in the image utilizing a computer vision algorithm and then, based at least in part on the identified identifiers and shadows, determine one or more anatomical distances. Example anatomical distances include, but are not limited to distances between the one or more markings on the transparent cover and a surface on which the shadow is located, a distance between an object in the image and a distal end of the visualization medical device, etc.

Further, it may be determined 208 whether an object for depth and/or size measuring is in the image. In some cases, this determination may be performed in an automated manner. Alternatively or additionally, the computing device may analyze the image using a computer vision algorithm and make a determination as to whether an object of interest exists in the image. Such computer vision algorithms may be well known in the art and/or other suitable computer vision algorithms.

When it has been determined an object for depth and/or size measuring is in the image, the method 200 may move to determining 210 a size measurement of the object. The size measurements of the object may be or may include a diameter of the object, a width of the object, a height of the object, a depth of the object, etc. The size measurements of the object may be determined in any suitable manner. In one instance, the computing device (e.g., the processor 112, such as a video processor, of the computing device 110 and/or other suitable computing devices) may analyze image data of the image to determine the size measurements based, at least in part, on the previously determined depth(s), but this is not required. If no object is identified or otherwise determined to be in the image, the method 200 may end 212. determinations of the tissue

Distances, depths (e.g., relative depths and/or other depths), and sizes of locations, areas, and/or objects in an image may be determined in any suitable manner using any suitable algorithm and/or technique. Example suitable algorithms or techniques for determining distances, depths, and/or sizes of locations of features in an image include, but are not limited to, shadow analysis techniques, structured lighting techniques, multi-point illumination techniques, time flight sensor techniques, leveraged optical aberration techniques, and/or other suitable techniques. Although this application is not limited to the examples therein, example measuring and/or sizing techniques are described in U.S. Patent Application Ser. No. 63/155,988, titled SCOPE MODIFICATIONS TO ENHANCE SCENE DEPTH INFERENCE filed on Mar. 3, 2021, which is hereby incorporated by reference in its entirety.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A visualization medical device, comprising:
an elongated shaft;
an imaging component extending through the elongated shaft, the imaging component having a lens and is configured to capture an image of an area exterior of the elongated shaft in a field of view of the lens; and
a transparent cover extending over the lens, the transparent cover configured to cause one or more identifiers to appear in the image.

2. The visualization medical device of claim 1, further comprising:
a lighting component extending through the elongated shaft, the lighting component is configured to light the area exterior of the elongated shaft.

3. The visualization medical device of claim 1, wherein the transparent cover includes a reticle in the field of view of the lens and the reticle appearing in a captured image is an identifier of the one or more identifiers.

4. The visualization medical device of claim 3, wherein the reticle is a fixed marking on the transparent cover.

5. The visualization medical device of claim 3, wherein the reticle comprises one or more liquid crystal display (LCD) elements actuatable to cause the one or more identifiers to appear in the image.

6. The visualization medical device of claim 3, further comprising:
a lighting component extending through the elongated shaft, the lighting component is configured to light the field of view of the lens and cause a shadow of the reticle to appears in the field of view of the lens.

7. The visualization medical device of claim 3, wherein the transparent cover is an extension extending distally from a distal end of the elongated shaft.

8. The visualization medical device of claim 3, wherein a distal end of the elongated shaft has one or more first mating components and a proximal side of the transparent cover has one or more second mating components configured to mate with the one or more first mating components.

9. The visualization medical device of claim 7, wherein at least one of the one or more first mating components is a female mating component and at least one of the one or more second mating components is a male mating component configured to mate with the female mating component.

10. The visualization medical device of claim 1, wherein the transparent cover is in direct contact with the lens.

11. The visualization medical device of claim 1, wherein the transparent cover is a first color at a first location and a second color at a second location.

12. A method for providing anatomical distance measurements using a medical visualization device, the method comprising:
applying a light through a transparent cover on an elongated shaft, the transparent cover having one or more markings and the light applied through the transparent cover causes a shadow to appear on a surface exterior of the transparent cover;
capturing an image through the transparent cover, the image including a view of the one or markings and at least a portion of the shadow; and
determining a measure of an anatomical distance in the image based on the view of the one or more markings and the portion of the shadow in the image.

13. The method of claim 12, further comprising:
adjusting a distal end of the elongated shaft to cause a target object to be within a field of view of a lens through which the image is captured.

14. The method of claim 12, further comprising:
determining a size measurement of an object in the image based on the measure of the anatomical distance.

15. The method of claim 12, wherein at least one of the one or more markings is an electronic component actuatable to be viewable through a lens used in capturing the image.

16. A visualization medical system, comprising:
an elongate tool having a transparent cover, the transparent cover having one or more markings thereon;
an imaging component configured to capture an image through the transparent cover, the image including one or more identifiers representative of at least one of the one or more markings on the transparent cover;
a video processor configured to analyze image data of the image that is received from the imaging component; and
wherein the video processor is configured to determine one or more measurements of an anatomical distance in the image based on the analysis of the image data.

17. The visualization medical system of claim 16, further comprising
a light source configured to apply light through the transparent cover and cause a shadow of the one or more markings to appear in the image.

18. The visualization medical system of claim 17, wherein the video processor is configured to determine the one or more measurements of the anatomical distance in the image based on the one or more markings in the image and the shadow of the one or more markings in the image.

19. The visualization medical system of claim 18, wherein the one or more measurements of the anatomical distance in the image include determining a distance between the one or more markings on the transparent cover and a surface on which the shadow is located.

20. The visualization medical system of claim 16, wherein the transparent cover is in direct contact with a lens of the imaging component.
